(19) United States
(12) Patent Application Publication
(10) Pub. No.: US 2024/0299635 A1
(43) Pub. Date: Sep. 12, 2024

(54) BREAST PUMP ASSEMBLY AND METHOD OF ALIGNING A BREAST PUMP ASSEMBLY

(71) Applicant: MAYBORN (UK) LIMITED, Tyne and Wear (GB)

(72) Inventors: Gerard HESELWOOD, Tyne and Wear (GB); Matthew GOSSINGTON, Tyne and Wear (GB)

(21) Appl. No.: 18/573,894

(22) PCT Filed: Jun. 23, 2022
(86) PCT No.: PCT/GB2022/051610

§ 371 Date: Dec. 22, 2023

(30) Foreign Application Priority Data
Jun. 23, 2021 (GB) 2109000.6

(51) Int. Cl. A61M 1/06

(52) U.S. Cl. CPC A61M 1/062; A61M 2205/587

(57) ABSTRACT

The present invention relates to a breast pump assembly, in particular a breast pump assembly suitable for aligning a nipple within a horn member of the assembly. More specifically, the breast pump assembly comprises a conduit comprising an opening adapted to receive a nipple of a user; and a light source arranged to project light towards the opening, such that, in use, light is projected onto the nipple of the user as the nipple is positioned into the opening. A further breast pump assembly comprises a breast receiving portion comprising an opening for receiving a breast of a user; and a light source arranged to project light from the opening to form a projected light region having an area corresponding to an area of the opening. The present invention also relates to a method of aligning a nipple within a breast pump assembly. Specifically, the method comprises projecting light from an opening of a nipple receiving portion of a breast pump assembly to form a projected light region, positioning the nipple of the user within the projected light region, and guiding the nipple into the opening of the nipple receiving portion whilst maintaining the nipple within the projected light region.

The present invention relates to a breast pump assembly, in particular a breast pump assembly suitable for aligning a nipple within a horn member of the assembly. The present invention also relates to a method of aligning a nipple within a breast pump assembly.

### INTRODUCTION

Breast pumps are used by nursing mothers to allow them to conveniently express their breast milk for storage and later feeding to infants. Such breast pumps stimulate milk expression by mimicking the action of a baby by applying suction to the breast. Suction applied to the breast in short or cyclical pulses provides efficient milk expression.

As used herein, the term breast pump assembly is understood to include at least a horn member of a breast pump, wherein a horn member is also known as a breast pump horn, breast pump shield or a breast shield.

In use the horn member of a breast pump assembly is positioned onto the breast so that at least the nipple is received within a narrowed opening or recess within the horn member. Once positioned, a negative pressure source is activated to apply suction to the breast via the narrowed opening or recess, thereby encouraging expression of breast milk. With the horn member positioned on the breast, a surface of the horn member preferably forms a seal with the user's breast to ensure that suction is maintained around the nipple without ambient air leaking in. The seal also prevents expressed milk leaking from the breast pump assembly instead of draining into a container.

A problem with known breast pump assemblies is the difficulty for a user to align the horn member as they position it on their breast because of the limited view of the user. This can result in the horn member being placed on the breast with the opening or recess misaligned with the nipple. Misalignment of the nipple leads to poor expression of milk due to the pump incorrectly or inefficiently applying suction. Misalignment may also cause discomfort or injury to the nipple through rubbing or pressure against the horn member. Nipple misalignment may not be immediately apparent to the user and may only be noticed from discomfort after extended use, or if the user finds that the volume of expressed milk is unexpectedly low.

The user has a limited view when positioning the horn member on their breast meaning that achieving a correct nipple alignment often relies on trial and error, potentially over a number of sessions using the breast pump. Such an approach risks ongoing discomfort to the user and inefficient expressing.

Often a user may need to use a breast pump while close to their infant so will not want to disturb the infant while attempting to positioning the horn member on their breast.

It is known in the art to use a horn member formed as separable components so that a first component, with an opening to receive the nipple, can initially be applied to the breast. The remainder of the horn member is then fixed to the first component to provide a breast pump assembly positioned on the breast for expressing milk. However, such an apparatus is difficult to use as it requires the user to assemble the remainder of the horn member without moving the first component once it is positioned on their breast. Furthermore, the user still has a limited view when positioning the first component onto the breast meaning some trial and error is unavoidable.

A further problem with known breast pump assemblies is that sealing of the breast against a surface of the horn member may be inadequate or incomplete, leading to reduced or inefficient suction from the negative pressure source, or leaking of expressed milk. This occurs particularly when a breast pump assembly includes a horn member which is an inappropriate size for the user's breast. Thus, the horn member surface may be too large, too small or have the wrong profile shape to receive and seal effectively against the breast. Furthermore, even though a horn member is initially a suitable size, a user's breast may alter as the infant grows, so that a horn member it may become unsuitable over time until such point as efficiency is reduced or expressed milk leaks.

Accordingly, it would be useful to provide a breast pump assembly and a method of aligning a nipple within a breast pump assembly which conveniently enables a user to correctly align their nipple for expressing breast milk using the assembly. In particular, it would be useful to provide a breast pump assembly that can more easily be positioned to express milk from the user's breast without discomfort and with improved efficiency.

It would also be useful to provide a convenient means to determine whether a horn member is suitably sized for a user's breast in order to provide an effective seal between the horn member and the breast.

### SUMMARY OF THE INVENTION

According to a first aspect of the invention, there is provided a breast pump assembly, including:
a conduit including an opening adapted to receive a nipple of a user; and
a light source arranged to project light towards the opening, such that, in use, light is projected onto the nipple of the user as the nipple is positioned into the opening.

As used herein, a light source which projects light, or which projects a beam of light, towards the opening projects light in a direction extending from the conduit towards its opening. That is, the light is projected from the light source along at least a portion of the conduit in a direction towards the opening. Light that is projected towards the opening is typically then projected from, that is through and beyond, the opening.

Aptly, the light source is arranged to project light from the opening. In this way, light is projected onto the nipple of the user as the nipple is aligned with, but exterior to, the conduit.

Aptly, the conduit is configured to focus light from the light source so as to project light from the opening within a predetermined region. That is, a significant proportion of the light projected towards, or from, the opening is concentrated into a beam of light. The beam of light thereby provides a region of projected light of increased intensity.

Aptly, the breast receiving portion is opaque. That is, the breast receiving portion is opaque to light emitted from the light source. More aptly, the breast receiving surface is textured. In these ways, the light projected from the light source is projected only from the opening. The light will thereby be projected with a predetermined region corresponding to the shape of the opening.

Aptly, the predetermined region has an area substantially equal to an area of the opening.

Aptly, the conduit is configured to form a shadow region surrounding the predetermined region.

Aptly, the conduit is configured such that light from the light source is projected from the opening in an axial direction.

Aptly, the opening is arranged perpendicular to a central longitudinal axis of the conduit.

Aptly, the conduit is enclosed by a wall. The wall forms an elongate tube extending between a first end and the opening. Preferably, the wall forms a closed terminus to the conduit at the first end.

Aptly, the light source is provided at the first end of the wall of the conduit.

Aptly, the light source is provided external to the conduit. That is, the light source is provided external to the wall enclosing the conduit. More aptly, the light source may be provided external to the first end of the wall.

Aptly, the light source is a source of visible light. That is, the light projected towards, or projected from, the opening of the breast pump assembly is visible light. More aptly, the light may be red light. Aptly, the light may have a wavelength within the range from 400 nm to 700 nm or, more aptly, within the range of from 650 nm to 700 nm.

Aptly, the breast pump assembly further includes a breast receiving portion extending from the opening of the conduit.

Aptly, the conduit and the breast receiving portion each include a wall having a refractive index such that a portion of light from the light source is refracted within the walls. In this way, the wall of the conduit and the wall of the breast receiving portion may have the same refractive index, or each wall may have a different refractive index.

Aptly, the refractive index of a wall is within a range from 1.3 to 1.7. More aptly, the refractive index of a wall is within a range 1.45 to 1.55.

Aptly, the wall of the conduit and the breast receiving portion is transparent or translucent. That is, the breast receiving portion is transparent to, or translucent to, light emitted from the light source.

Aptly, light refracted within the walls is projected from a perimeter edge of the breast receiving portion.

Aptly, the breast receiving portion flares outwardly from the opening of the conduit to a perimeter edge.

Aptly, the breast receiving portion is integrally formed with the conduit.

Aptly, the light source is adapted to selectively project light.

Aptly, the conduit includes a negative pressure supply port.

Aptly, a breast pump assembly further includes a negative pressure source fluidly coupled to the negative pressure supply port such that, when activated, the negative pressure source provides a negative pressure to the conduit.

Aptly, the negative pressure source is configured to be selectively activated, and wherein the light source is configured to deactivate when the negative pressure source is activated.

According to a second aspect of the invention, there is provided a breast pump assembly, including:
a breast receiving portion including an opening for receiving a breast of a user;
and a light source arranged to project light from the opening to form a projected light region having an area corresponding to an area of the opening.

Aptly, the breast receiving portion includes a perimeter edge around the opening, wherein the light source and the breast receiving portion are configured such that a portion of light from the light source is projected from the perimeter edge.

Aptly, the breast receiving portion includes a wall having a refractive index such that a portion of light from the light source is refracted within the walls and projected from the perimeter edge.

Aptly, the perimeter edge is configured to form a ring shaped shadow around the projected light region.

According to a third aspect of the invention, there is provided a method of aligning a nipple within a breast pump assembly, the method including:
projecting light from an opening of a nipple receiving portion of a breast pump assembly to form a projected light region,
positioning the nipple of the user within the projected light region,
guiding the nipple into the opening of the nipple receiving portion whilst maintaining the nipple within the projected light region.

Aptly, the method further includes focussing the light projected from the opening within a predetermined region.

Aptly, the predetermined region has an area substantially equal to an area of the opening of the nipple receiving portion.

According to a fourth aspect of the invention, there is provided a breast pump assembly including:
a conduit including an opening adapted to receive a nipple of a user; and
a light source arranged to project a beam of light from the opening such that, in use, the beam of light is focussed onto the nipple of the user as the nipple is positioned into the opening.

Aptly, the conduit includes a central longitudinal axis and the beam of light is focussed along the central longitudinal axis.

Aptly, the beam of light is focussed within a predetermined region.

Aptly, the breast receiving portion is opaque. That is, the breast receiving portion is opaque to light emitted from the light source. More aptly, the breast receiving surface of the breast receiving portion is textured. In these ways, the light projected from the light source is projected only from the opening. The light is be projected with a predetermined region corresponding to the shape of the opening.

Aptly, the conduit has a diameter in a range of from 10 mm to 40 mm, more aptly in a range from 20 mm to 32 mm.

Aptly the conduit includes a first conduit portion with a first diameter and second conduit portion with a second diameter, wherein the second diameter extends to the opening. More aptly, the second diameter is greater than the first diameter. In this way, the first conduit portion focusses the beam of light within a narrower predetermined region.

Aptly, the first diameter is in a range of from 10 mm to 20 mm. Aptly, the second diameter is in the range from 20 mm to 40 mm, preferably in the range 24 mm to 28 mm. In this way, while the second diameter is sized to receive the user's nipple, the first conduit portion provides a more narrowly focussed beam of light.

Aptly, the predetermined region has an area substantially equal to an area of the opening.

Aptly, the predetermined region has a cross-sectional area determined at a distance from the opening. That is the cross-sectional area of a beam of light may be determined after it has been projected from the opening in a plane perpendicular to the beam of light. Aptly, the cross-sectional area of the beam of light at a distance of 50 mm from the opening is in the range from 20 mm² to 8000 mm², and more aptly in the range from 100 mm² to 5000 mm², and yet more aptly, in the range from 400 mm² to 1500 mm².

Aptly, the cross-sectional area of the predetermined region is circular. The cross-sectional area may have a diameter at a distance 50 mm from the opening of less than or equal to 100 mm, more aptly of less than or equal to 60 mm and yet more aptly of less than or equal to 40 mm. The cross-sectional area may have a diameter at a distance of 50 mm from the opening in the range of from 5 mm to 100 mm, more aptly in the range 10 mm to 60 mm, and yet more aptly in the range from 25 mm to 35 mm.

Aptly, the conduit is configured such that light from the light source is projected from the opening in an axial direction.

Aptly, the opening is arranged perpendicular to a central longitudinal axis of the conduit.

Aptly, the light source is provided external to the conduit.

Aptly, the beam of light is focussed using a lens assembly. More aptly, the lens assembly includes a Fresnel-type lens.

Aptly, the conduit of the breast pump assembly includes a wall. A lens assembly may be integrally formed with a portion of the wall.

Aptly, one or both of the lens assembly and the wall may be a transparent polymer material. More aptly, the polymer may be a polycarbonate, an acrylonitrile butadiene styrene, a nylon or an acrylic polymer, or a mixture thereof. In a preferred example, the polymer of a lens assembly is polycarbonate.

Aptly, a breast pump assembly further includes a breast receiving portion, wherein the breast receiving portion extends from the opening of the conduit.

Aptly, the conduit and the breast receiving portion each include a wall having a refractive index such that a portion of light from the light source is refracted within the walls. More aptly, the refractive index is within a range from 1.3 to 1.7.

Aptly, light refracted within the walls is projected from a perimeter edge of the breast receiving portion.

Aptly, the breast receiving portion flares outwardly from the opening of the conduit to a perimeter edge.

Aptly, the breast receiving portion is integrally formed with the conduit.

Aptly, the light source of the breast pump assembly is adapted to selectively project light. In certain examples, the light source may be selectively switched on or off by a user of the breast pump assembly. In this way light source may be conveniently used to align the nipple in the opening ready for expressing milk and, optionally, deactivated during expressing. Alternatively, the light source may be configured to automatically deactivate when the negative pressure source is activated.

Aptly, the conduit includes a negative pressure supply port. More aptly, the breast pump assembly further includes a negative pressure source fluidly coupled to the negative pressure supply port such that, when activated, the negative pressure source provides a negative pressure to the conduit.

Aptly, the negative pressure source is configured to be selectively activated. Optionally, the light source may be configured to deactivate when the negative pressure source is activated.

According to a fifth embodiment of the invention, there is provided a breast pump assembly, including:
a breast receiving portion including an opening for receiving a breast of a user, wherein the opening includes a longitudinal axis extending therethrough; and
a light source arranged to project light from the opening to focus a beam of light along the longitudinal axis.

Aptly, the breast receiving portion includes a perimeter edge around the opening, wherein the light source and the breast receiving portion are configured such that a portion of light from the light source is projected from the perimeter edge.

Aptly, the breast receiving portion includes a wall having a refractive index such that a portion of light from the light source is refracted within the walls and projected from the perimeter edge.

According to a sixth embodiment of the invention, there is provided a method of aligning a nipple within a breast pump assembly, the method including:
projecting light from an opening of a nipple receiving portion of a breast pump assembly,
focussing the light into a beam of light;
positioning a nipple of the user within the beam of light,
guiding the nipple into the opening of the nipple receiving portion whilst maintaining the nipple within the beam of light.

Aptly, the method further includes focussing the light projected from the opening into a beam of light within a predetermined region with a cross-sectional area. The cross-sectional area may be determined at a distance from the opening. That is the cross-sectional area of a beam of light may be determined after it has been projected from the opening in a plane perpendicular to the beam of light. Aptly, the cross-sectional area of the beam of light at a distance of 50 mm from the opening is in the range from 20 mm² to 8000 mm², and more aptly in the range from 100 mm² to 5000 mm², and yet more aptly, in the range 400 mm² to 1500 mm².

Aptly, the cross-sectional area of the predetermined region is circular. The cross-sectional area may have a diameter at a distance 50 mm from the opening of less than or equal to 100 mm, more aptly of less than or equal to 60 mm and yet more aptly of less than or equal to 40 mm. The cross-sectional area may have a diameter at a distance of 50 mm from the opening in the range of from 5 mm to 100 mm, more aptly in the range 10 mm to 60 mm, and yet more aptly in the range from 25 mm to 35 mm.

Aptly, the beam of light is a parallel, or a substantially parallel beam of light. That is the beam of light may be focussed so that it has a consistent cross-sectional area at increasing distances from opening.

Aptly, the conduit includes a central longitudinal axis so that the step of focussing the light provides a beam of light focussed along the central longitudinal axis.

Certain examples allow the user to easily see if their nipple is centred in the middle of the opening as the horn member is positioned on the breast. Certain examples provide an illuminated guide to enable a user to align the opening of a breast pump assembly for expressing breast milk. In particular, the user can align the opening without contacting the breast and also avoiding trial and error.

Certain examples provide an illuminated guide to enable a user to determine the appropriate sizing of a horn member. In particular, the user can determine the appropriate size opening for their nipple or can determine the appropriate size breast receiving portion for their breast. Thus, the user can determine appropriate sizes without contacting the breast and also avoiding trial and error.

Certain examples provide an illuminated guide to enable a user to use a breast pump assembly in a dark or dimly lit environment so as to express milk without disturbing their infant.

Certain examples have the advantage that one, or both, of the conduit or the lens assembly acts as a lens to focus light projected towards, or from, the opening. The light projected towards, or from, the opening may provide a projected light region which a user uses to guide the horn member onto the breast, while ensuring that the nipple is centred in the projected light region. In this way the nipple will be conveniently positioned within the opening as the user guides the horn member onto the breast.

Certain examples have the advantage that one, or both, of the conduit or lens assembly projects a beam of light towards, or from, the opening. The beam of light projected towards, or from, the opening may provide a projected light region which a user uses to guide the horn member onto the breast, while ensuring that the nipple is centred in the projected light region.

The beam of light, or the projected light region, may provided an illuminated area of comparatively increased light intensity than would be achieved otherwise. Thus, beam of light allows the opening of the breast pump assembly to be aligned to a user's nipple even in bright lighting conditions.

Certain examples have the advantage that light which enters the walls of the horn member will be refracted through its wall and will exit, that is be projected from, an edge of the horn member. In this way, refracted light forms a further projected light region. The further projected light region may be significantly brighter than a neighbouring shadow region, or a neighbouring projected light region.

It will be appreciated that any of the features described above in relation to the first aspect of the invention may apply equally to the other aspects of the invention. That is, any features described above under the first aspect may be combined with the breast pump assembly of the second aspect or with the method of the third aspect and so on. Also, any features described above under the assemblies of the first, second, fourth or fifth aspect may be combined with the method of both the third and sixth aspect.

### BRIEF DESCRIPTION OF THE DRAWINGS

Embodiments of the invention are now described, by way of example only, with reference to the accompanying drawings, in which:

FIG. 1 shows a cross-sectional view of a breast pump assembly;

FIG. 2 shows a cross-sectional view of a further breast pump assembly;

FIG. 3 shows schematic view of a breast pump assembly including the assembly of FIG. 1 and further components;

FIG. 4 shows perspective view of another breast pump assembly;

FIG. 5 shows a cross-sectional view of another breast pump assembly;

FIG. 6 shows a cross-sectional view of another breast pump assembly;

FIG. 7 shows a method of aligning a nipple within a breast pump assembly;

FIG. 8 shows a cross-sectional view of a further breast pump assembly including a lens assembly;

FIG. 9 shows a) a perspective view, and b) a cross-sectional view, of the lens assembly of the breast pump assembly of FIG. 8;

FIG. 10 shows a cross-sectional view overlaid with a computer-modelled ray tracing diagram of the breast pump assembly of FIG. 2; and

FIG. 11 shows a cross-sectional view, overlaid with a computer-modelled ray tracing diagram, of the breast pump assembly of FIG. 8.

In the drawings, like reference numerals refer to like parts.

### DETAILED DESCRIPTION

Certain terminology is used in the following description for convenience only and is not limiting. The words ‘internal’ and ‘external’ designate directions in the drawings to which reference is made and are with respect to the described component when assembled and mounted. The words ‘inner’ and ‘outer’ refer to directions toward and away from, respectively, a designated centreline or a geometric centre of an element being described (e.g. central axis), the particular meaning being readily apparent from the context of the description.

Further, unless otherwise specified, the use of ordinal adjectives, such as, ‘first’, ‘second’, ‘third’ etc. merely indicate that different instances of like objects are being referred to and are not intended to imply that the objects so described must be in a given sequence, either temporally, spatially, in ranking or in any other manner.

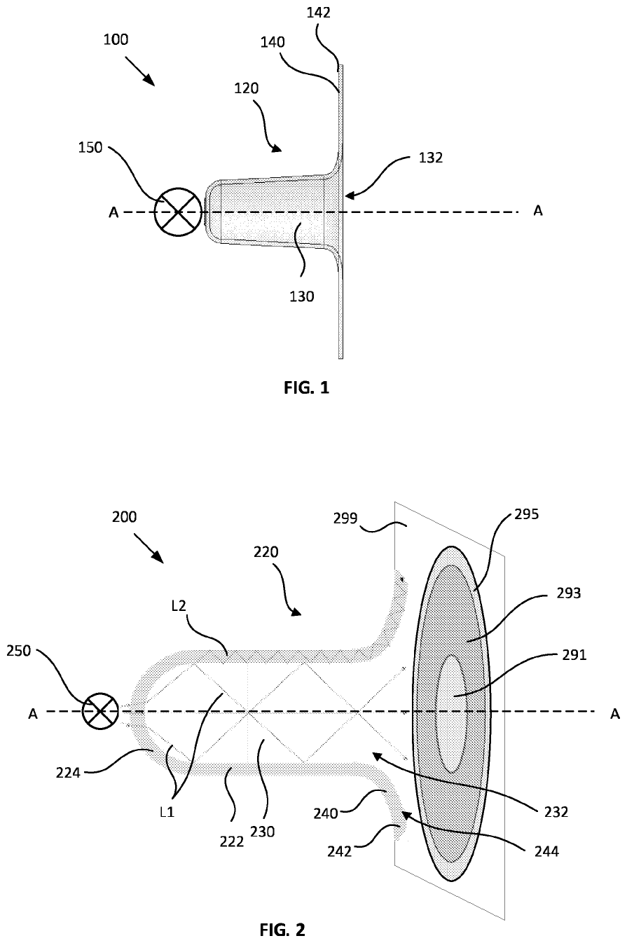

Referring now to FIG. 1, there is shown a breast pump assembly 100 including a conduit 130 and a light source 150. The conduit 130 includes an opening 132 adapted to receive a nipple of a user. That is, the opening 132 is sized and shaped such that a user may position their nipple therethrough such that the nipple is received in the conduit.

The light source 150 is arranged to project light towards the opening 132, such that, in use, light is projected onto the nipple of the user as the nipple is positioned into the opening 132. In this way, as the user positions their nipple into the opening, the light projected from the light source is directed onto the nipple to provide a visual guide to the user, enabling the user to more easily align their nipple with the opening 132.

The light source 150 may be arranged to project light towards and from the opening 132. As described herein, the light source 150 thereby projects light in a direction from the conduit, towards and then beyond the opening 132. As such, the light may be projected onto the nipple of the user, when the nipple is at a position distal from the opening 132, thereby enabling the user to more easily align their nipple with the opening 132.

The conduit 130 is configured to focus light projected from the opening 132 within a predetermined region. That is, the conduit 130 is sized and shaped such that light from the opening provides a projected light region. The projected light region may have an area substantially equal to the area of the opening 132. As such, the nipple can be aligned with the opening even when at a position distal therefrom.

The conduit 130 may be further configured to form a shadow region surrounding the predetermined region. In this way, the conduit 130 may be further configured to define an outer perimeter to the projected light region such that the shape and size of the outer perimeter is provided by the shadow region. The outer perimeter may therefore be clearly defined so that boundary to the predetermined region it is distinct.

The conduit 130 may be further configured such that light from the light source 150 may be projected from the opening 132 in an axial direction. That is, the conduit 130 may extend along a longitudinal axis A and light may be projected from the opening in substantially the same direction in which the conduit 130 extends.

The breast pump assembly 100 includes a horn member 120 with a breast receiving portion 140 extending from the opening 132 of the conduit 130. The breast receiving portion 140 engages the user's breast as the nipple is received into the opening 132. The breast receiving portion 140 may thereby sealingly engage the user's breast such that, during use, suction from within the conduit 130 can be maintained around nipple without ambient air leaking in, and to prevent expressed milk leaking from the opening 132. The breast receiving portion 140 may extend from the opening 132 of the conduit 130 to a perimeter edge 142 of the horn member 120.

To use the breast pump assembly 100, the horn member 120 is fluidly connected to a negative pressure source to enable suction to be applied to a user's breast within the conduit in order to express milk. The breast pump assembly may thus include the negative pressure source, which may be a manual breast pump, in which the negative pressure source is actuated by a user so that suction is generated by a manual action of a user. For example, a user may actuate a mechanical pump or deform a resilient container.

Alternatively, the breast pump assembly may include an electric breast pump, in which the negative pressure source is a suitable electrical apparatus, such as an electromechanical pump.

When using the breast pump assembly 100 to express milk, the user will first correctly position the horn member 120 so as to align the nipple with the opening 132. To help correctly position the horn member the light source 150 is activated, such that light is projected from the opening 132 as a first projected light region. That is, light is projected from the opening 132 within the predetermined region. The user brings the horn member 120 towards the breast and aligns the horn member 120 so that the nipple of the breast is within the projected light region. In this way, the user aligns the horn member 120 so that the nipple is illuminated by light projected from the opening 132.

Whilst maintaining the nipple within the projected light region, the user then moves the horn member 120 towards the breast until the nipple is within the opening 132. The user then continues moving the horn member 120, guiding the nipple into the opening 132, until the breast engages the breast receiving portion 140. Thus, the user can easily guide the nipple into the centre of the opening 132 ready for expressing milk. The nipple is thereby correctly positioned within the conduit 130 so as to receive suction from a negative pressure source and the breast is comfortably engaged with the breast receiving portion 140 so as to form an effective seal.

Referring now to FIG. 2, there is shown a schematic image of a further example breast pump assembly 200. Where the features are the same as a previous example, the reference numbers are the same, other than the initial digit is a “2”. The breast pump assembly 200 is substantially the same as breast pump assembly 100 of FIG. 1, other than the breast receiving portion 240 flares from the opening 232. That is, the breast receiving portion 240 flares outwardly from the opening 232 so that a perimeter edge 242 is radially and axially spaced from the opening 232.

As the breast receiving portion 240 flares from the opening 232, there is provided a concave breast receiving surface 244. In this way, the horn member 220 extends from the opening 232 to form a breast receiving surface 244 around the opening 232. In alternative arrangements, the breast receiving portion 240, or a part thereof, may be configured to provide any suitable breast receiving surface to engage the breast in a comfortable manner and/or to create an effective seal. In this way, the breast receiving surface may be a recessed cone, dome, hemisphere or any hybrid shape thereof. The concave surface of the breast receiving surface may be symmetrically-shaped or may be asymmetrically-shaped.

Yet further, a breast receiving portion may alternatively be arranged perpendicular to the longitudinal axis A of the conduit so that the breast receiving portion extends radially away from the opening. In this way, the breast receiving portion of the horn member forms a substantially flat, annular breast receiving surface with which to engage the breast.

The breast receiving portion 240 is aptly formed integrally with the conduit 230 such that the horn member 220 is formed as a single part.

The conduit 230 of the breast pump assembly 200 is enclosed by a wall 222 of the horn member 220. The wall 222 forms an elongate tube extending between a first end 224 and the opening 232. At the first end 224, the wall 222 forms a closed terminus to the conduit 230.

In the example shown in FIG. 2, the light source 250, first end 224 and opening 232 are each arranged on the axis A. Alternatively, the conduit may curve away from the longitudinal axis A as it extends from the opening 232 to the first end 224.

The wall 222 is configured to surround a user's nipple once it is received into the opening 232 of the conduit 230. In this way, the conduit 230 is provided with a cylindrical cross-section. In alternative configurations, the conduit may be provided with other suitable cross-sectional shapes, for example an ellipse. The conduit may have any cross-sectional and axial shapes appropriate to surround a user's nipple while providing comfort as negative pressure is applied to express milk. The length of the conduit, in other words the distance that the conduit extends between the first end 224 and the opening 232, may be selected so as to provide space to allow the nipple to stretch or deform when negative pressure is applied. Furthermore, the conduit may taper along at least a portion of its axial length. At least a portion of the conduit may taper so as to narrow or widen as it extends from the opening.

In certain examples, the conduit may be surrounded by a wall without a first end. That is, the conduit may extend from the opening into which the nipple is positioned to a further opening at an opposing end. Aptly, the first end or the further opening may include a port to fluidly connect the conduit to, for example, a negative pressure source or a container.

The light source 250 of the breast pump assembly 200 is arranged external to the first end 224 of the wall 222 surrounding the conduit 230.

The horn member 220 is transparent to light emitted by the light source 250. Preferably, the member horn 220 may be formed of plastic, so as to also minimise the weight of the breast pump assembly. In the example shown in FIG. 2, the horn member 220 is formed of a transparent polypropylene.

The transparent material of a horn member may have any suitable refractive index. In the example shown in FIG. 2, the transparent polypropylene material has a refractive index of 1.5. In this example, the horn member 220 includes a breast receiving portion 240 integrally formed with the wall 222 of the conduit 230. As such, both the breast receiving portion 240 and the wall 222 are formed of a common material, in this case a common transparent polypropylene material with a refractive index of 1.5.

A first portion of light L1 emitted by the light source 250 passes through the wall 222 and into the conduit 230. Light which enters the conduit 230 is projected along the conduit 230 due to the reflective nature of the surface of the wall 222. Consequently, the first portion of light L1 is reflected along the conduit 230 towards the opening 232.

The light L1 is projected from the opening 232. Due to the cylindrical shape of the wall 222, light projected from the opening 232 is projected in a direction corresponding to the longitudinal axis A. In this way, a surface 299 positioned near the opening 232 in the direction of the longitudinal axis A is illuminated by light projected therefrom. The light projected from the opening 232 provides a first projected light region 291 having a cross-sectional area substantially the same size and shape as the opening 232. The first projected light region 291 corresponds to light focussed by the conduit 230.

The light projected from the opening 232 also provides a shadow region 293. In the example of FIG. 2, the shadow region 293 surrounds the first projected light region 291. The shadow region 293 is a region with a comparatively reduced level of illumination from the light source 250. In other words, by reflecting and focussing light along it, the conduit 230 also provides a shadow region 293 to define the outer edge of the first projected light region 291.

A second portion of light L2 entering the wall 222 from the light source 250 is refracted within the wall 222 itself. Thus, due to its refractive properties, a second portion of the light L2 from the light source 250 is refracted within the horn member 220 in a direction away from the light source 250.

In the example shown in FIG. 2, the light source 250 is positioned relative to the horn member 220 so that refracted light is initially refracted within the wall 222 of the conduit 230 towards the opening 232. Due to the flared shape of the breast receiving portion 240 of the horn member 220, the second portion of light L2 is refracted within the breast receiving portion 240 towards its perimeter edge 242. The second portion of light L2 is then projected from the perimeter edge 242.

The shape of the breast receiving portion 240 provides a substantially annular, or ring-shaped perimeter edge 242, oriented in a plane perpendicular to the longitudinal axis A. The second portion of light L2 is projected from the perimeter edge 242 in a direction away therefrom. In this way, a surface 299 positioned near the perimeter edge 242 is illuminated by light projected as a second projected light region 295. The second projected light region 295 is substantially the same shape as the perimeter edge 242. In the example shown in FIG. 2, the second projected light region 295 is an annular projected light region.

The second projected light region 295 defines an outer edge to the shadow region 293. In this way, the horn member 220 is configured to form concentrically arranged first and second projected light regions 291, 295. The first and second projected light regions 291, 295 are separated by a shadow region 293.

In an alternative arrangement, the breast receiving portion may be configured so as not to provide a second projected light region. For example, perimeter edge may be shape to disperse the second portion of light L2 without forming a distinctive projected light region.

A user desiring to use the breast pump assembly 200 to express milk positions the horn member 220 so as to align the nipple in substantially the same manner as when using the breast pump assembly 100 shown in FIG. 1. Thus, in use, the surface 299 illuminated by light projected from horn member 220 is a user's breast. The user aligns their nipple within the first projected light region 291. Maintaining the nipple within the first projected light region 291, the user moves the horn member 220 towards the breast until the nipple is within the opening 232. The user then continues moving the horn member 220, guiding the nipple into the opening 232, until the breast engages the breast receiving portion 240.

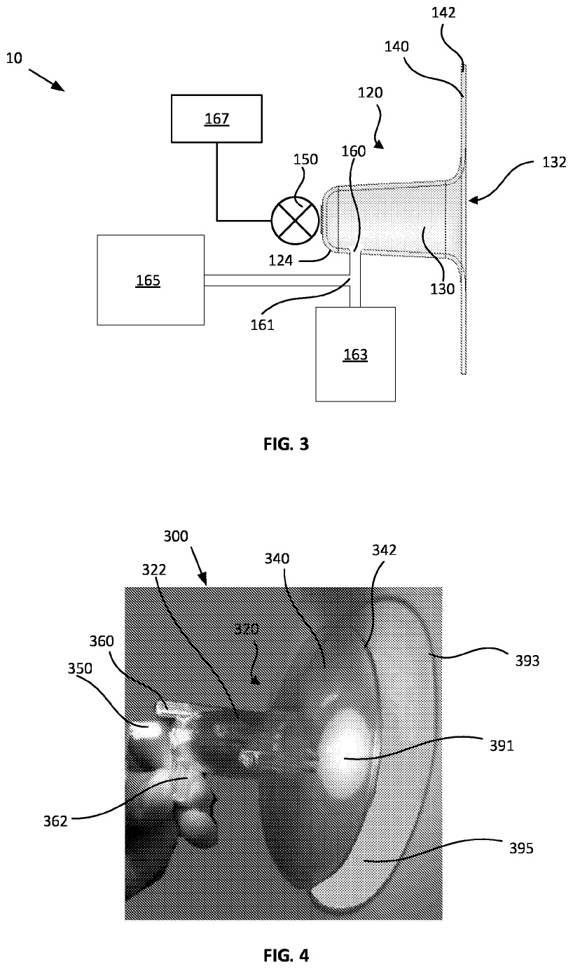

Referring now to FIG. 3, there is shown a representation of a fully assembled breast pump assembly 10. The breast pump assembly 10 includes all the same features of the breast pump assembly 100 of FIG. 1, including the conduit 130 and opening 132 adapted to receive a nipple of a user, and a light source 150 arranged to project light towards the opening 132.

Furthermore, in the breast pump assembly 10 shown in FIG. 3, the conduit 130 is enclosed by a wall of the horn member 120. In this way, the wall forms an elongate tube extending between a first end 124 and the opening 132.

A port 160 is provided proximal the first end 124. A connector 161 fluidly connects the port 160 to a negative pressure source 165, which is adapted to generate a negative pressure for applying suction to a nipple received within the conduit 130. The connector 161 also fluidly connects the port 160 to a container 163 arranged to collect milk expressed into the conduit 130. Any suitable connector, negative pressure source and container as are known in the art may be fluidly connected to the port 160 of the breast pump assembly 10 to provide a breast pump capable of expressing breast milk.

A controller 167 is operably connected to the light source 150. The controller 167 is adapted to selectively activate and deactivate the light source 150. The controller 167 is manually operated, for example by the user pressing a button, so that the user manually may activate the light source 150 when using the breast pump assembly 10. When activated, the light source 150 emits light to allow the user to align their nipple within the opening 130 as described above. Once the nipple is aligned, the user deactivates the light source 150, for example by pressing the button a second time.

Alternatively, the controller may be configured to activate the light source only for a predetermined period of time, thereby removing the need for the user to press the button a second time to deactivate the light source. Optionally, where the breast pump is an electrical breast pump, the controller may also be operably connected to the negative pressure source. In these ways, when the light is deactivated, either manually or after a predetermined time period, then the controller may automatically activate the negative pressure source to begin milk expression. Thus, the horn member can be readily connected with the negative pressure source such that the breast pump assembly is simple for the user to operate.

A breast pump assembly such as the examples described above, may be adapted to provide other arrangements of shadow regions and projected light regions by any suitable configuration of features of the horn member, such as the opening, the breast receiving portion and its perimeter edge. Thus, the horn member may be adapted to provide a range of shape, size and number of projected light regions and shadow regions to align a nipple within a breast pump assembly.

Referring now to FIG. 4, there is shown a further breast pump assembly 300. Where the features are the same as a previous example, the reference numbers are the same, other than the initial digit is a “3”. The conduit and opening are substantially the same as the example shown in FIG. 1 with the exception that the breast receiving portion 340 is concavely shaped so that the breast receiving surface is a symmetrically-shaped recessed dome.

The wall 322 of the conduit is provided with first and second ports 360, 362. In contrast to the example shown in FIG. 3, the first and second ports 360, 362 enable the conduit to be fluidly connected independently to a negative pressure source and to a container. Thus, the first port 360 is adapted to fluidly connect the conduit to a negative pressure source and the second port 362 is adapted to fluidly connect the conduit to a container.

With the light source 350 activated, light passing through the wall 322 and entering the conduit is reflected along the conduit and projected from the opening. The reflected light provides a first projected light region 391 having a cross-sectional area substantially the same size and shape as the opening to the conduit. The first projected light region 391 corresponds to light focussed by the conduit.

The light source 350 is positioned external to the horn member 320. Due to its position, a portion of the light from the light source 350 is directly received by the external surface of the breast receiving portion 340. The external surface is a surface of the breast receiving portion 340 opposing the breast receiving surface. The breast receiving portion 340 is formed of a transparent material, enabling light from the light source 350 to pass directly through the breast receiving portion 340 to form a second projected light region 395. The second projected light region 395 surrounds and directly bounds the outer edge the first projected light region 391. The first projected light region 391 is contiguous with the second projected light region 395. The first projected light region 391 may generally be brighter relative to the second projected light region 395.

In certain examples, a portion of the horn member, such as a portion of the breast receiving portion, may provide a blocking portion to the light emitted by the light source 350. In the example shown in FIG. 4, the perimeter edge 342 provides a blocking portion so as to block light from the light source 350. That is, although the breast receiving portion 340 is formed from a transparent material, the perimeter edge 342 has optical properties which differs to the remainder of the breast receiving portion 340. Consequently, the perimeter edge 342 acts as a barrier to light received directly from the light source 350.

The perimeter edge 342 acts as a blocking portion as to form a shadow region 393 with the projected light. In the example shown, the shadow region 393 surrounds the second projected light region 395. The outer edge of the second projected light region 395 is thereby defined by the shadow region 393.

In this way, the breast pump assembly 300 is configured to form concentrically arranged first and second projected light regions 391, 395. The first and second projected light regions 391, 395 are encompassed by a shadow region 393.

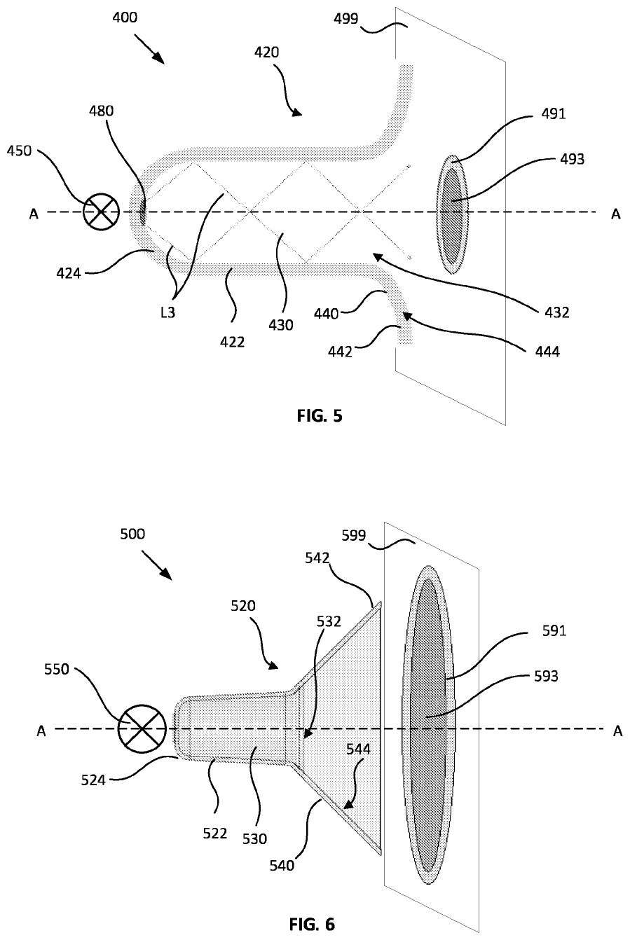

The properties or structure of the horn member may be adapted in a number of ways to provide to provide a blocking portion. Further, non-limiting examples are set out in the descriptions of FIGS. 5 and 6 below. Thus, the dimensions, shape and refractive properties of a horn member may be selected so as to determine the proportion of the emitted light passing through and refracting within its walls or a within a portion of its wall. In other words, the horn member may be configured to determine the proportion of light entering the wall that will pass through the wall and enter the conduit, and the proportion of light that is refracted within the horn member. In this way, by appropriate selection of the properties of the wall, the illumination levels of any projected light region can be controlled.

Referring now to FIG. 5, there is shown a further breast pump assembly 400. Where the features are the same as a previous example, the reference numbers are the same, other than the initial digit is a “4”. The breast pump assembly 400 is substantially the same as the example shown in FIG. 2 with the exception that the conduit 430 further includes a blocking portion 480.

The blocking portion 480 is adapted to block or limit a portion of the light entering the conduit 433 so as to cast a shadow within the light L3 projected towards the opening 432. Consequently, the light projected from the opening 432 forms an annular projected light region 491. The annular projected light region 491 surrounds a central shadow region 493 formed by the shadow cast by the blocking portion 480. The shadow region 493 is projected along the longitudinal axis A of the conduit 430 with annular projected light region 491 extending concentrically therearound.

In the example shown, the blocking portion 480 is provided as an opaque circular mask on the inner surface of the first end 424 of the wall 422 of the conduit 430. However, any suitable blocking portion may be provided within the wall 422, or on its inner or outer surfaces. Alternatively, a blocking portion may be positioned to be spaced away from the wall so that it is arranged, for example, within the conduit, or positioned external to the wall. Consequently, a blocking portion may be any object, such as a mask or an opaque body, capable of acting as a barrier to a portion of light received by the horn member. In particular, in the example shown in FIG. 5, the blocking portion 480 acts as a barrier to light that would otherwise be projected along the conduit 430.

With the light source 450 activated, the annular projected light region 491 is projected from the opening 432. The user brings the horn member 420 towards the breast and aligns the horn member 420 so that the nipple is within the annular projected light region 491. Keeping the nipple within the annular projected light region 491, the user moves the horn member 420 towards the breast, thereby positioning the nipple within the opening 432. The user continues moving the horn member 420, guiding the nipple into the opening 432, until the breast engages the breast receiving surface 444 of the breast receiving portion 440.

The nipple is thereby correctly positioned within the conduit for applying suction to express milk, and the breast is comfortably engaged with the breast receiving surface 444 so as to form an effective seal.

Advantageously, with the light source 450 activated, the annular projected light region 491 may also be used to determine whether the opening 432 of the horn member 420 is correctly sized for the user's nipple. Thus, with their nipple within the annular projected light region 491, the user is able to determine from the position of the projected light whether the opening 432 is a correct size for their nipple. If the opening 432 is an incorrect size, for example if it is too small for the nipple, the horn member can be changed to an alternative and its suitability checked again. Using the projected light region, each horn member can be easily checked for suitability without contacting the nipple and without trial and error or a period of inefficient use.

Referring now to FIG. 6, there is shown a further breast pump assembly 500. Where the features are the same as a previous example, the reference numbers are the same, other than the initial digit is a “5”. The breast pump assembly 500 is substantially the same as the example shown in FIG. 1 with the exception that the breast receiving portion 540 of the horn member 520 flares from the opening 532 so that the breast receiving surface 544 forms a symmetrically-shaped recessed cone. Due to the shape of the horn member 520, the wall of the breast receiving portion 540 itself acts as a blocking portion to light from the light source 550.

With the light source 550 activated, light is refracted within the horn member 520 and projected from the perimeter edge 542 to form a first projected light region 591. The first projected light region 591 has a cross-sectional area substantially the same size and shape as the perimeter edge 542 of the horn member 520. That is, the first projected light region 591 forms an annular ring of projected light.

In use, with the light source 550 activated, a user brings the horn member 520 towards their breast so that the first projected light region 591 is projected onto their breast. The user can then determine whether the horn member 520 of the breast assembly 500 is the correct size for them, or if they need a smaller or larger size of the horn member. Additionally, if a user notices discomfort or reduced amounts of milk when expressing, they are able to check whether a horn member is still the optimum size or should be changed for an alternative size. Thus, a user can readily and easily determine the appropriate size of horn member without using trial and error of multiple parts.

Optionally, the first end of the wall 522 includes a further blocking member which restricts light from entering the conduit 530 so that the only light projected by the horn member 520 is light projected from the perimeter edge 542. However, the further blocking member may not be necessary so long as the shape of the horn member 520 provides a distinct projected light region with a cross-sectional area substantially the same size and shape as the perimeter edge 542 of the horn member 520.

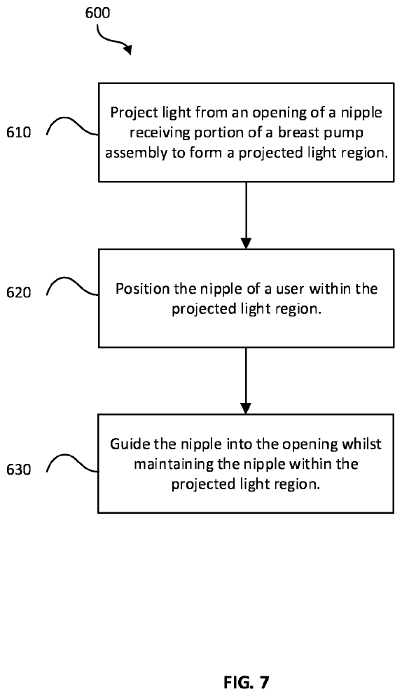

Referring now to FIG. 7, there is shown flow chart illustrating a method 600 of aligning a nipple within a breast pump assembly. The method may be carried out using any suitably adapted breast pump assembly such as, for example, any of the assemblies as described herein.

A first step 610 of the method 600 includes projecting light from an opening of a nipple receiving portion of a breast pump assembly to form a projected light region. In the example breast pump assemblies described above, a nipple receiving portion may be a conduit of a horn member although the nipple receiving portion may be any other conduit, recess or passage of a breast pump assembly suitably configured to receive a nipple.

A second step 620 includes positioning the nipple of the user within the projected light region. That is, the nipple or the breast pump assembly is positioned so that the projected light region illuminates the nipple.

A third step 630 includes guiding the nipple into the opening of the nipple receiving portion whilst maintaining the nipple within the projected light region. In this way, the projected light enables the user to easily see if their nipple is centred in the middle of the opening as it is positioned within a breast pump assembly. Thus, when using a breast pump assembly for expressing the user can readily and easily determine that their nipple is correctly positioned so that milk can be expressed effectively and in a comfortable manner.

In certain examples, the method may include a further step of focussing the light projected from the opening within a predetermined region. Optionally, the predetermined region may have an area substantially equal to an area of the opening of the nipple receiving portion

Consequently, the nipple receiving portion, for example a conduit of the nipple receiving portion, may be sized and shaped to focus light to provide the projected light region. As such, projected light extends from the opening in a controlled manner such that the method enables the nipple to be aligned with the opening even when at a position distal therefrom.

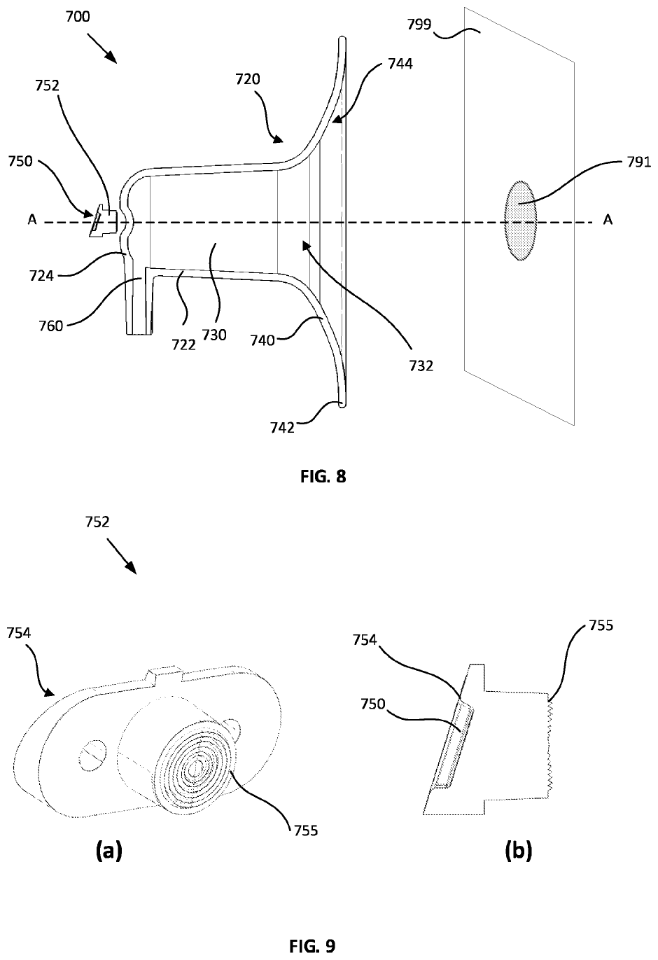

Referring now to FIG. 8, there is shown a further example breast pump assembly 700. Where the features are the same as a previous example, the reference numbers are the same, other than the initial digit is a “7”. The breast pump assembly 700 is substantially the same as breast pump assembly 700 of FIG. 2, including a breast receiving portion 740 which flares outwardly from the opening 732 so that a perimeter edge 742 is radially and axially spaced from the opening 732.

The conduit 730 of the breast pump assembly 700 is enclosed by a wall 722 of the horn member 720. The wall 722 forms an elongate tube extending between a first end 724 and the opening 732. At the first end 724, the wall 722 forms a closed terminus to the conduit 730.

The wall 722 is configured to surround a user's nipple once it is received into the opening 732 of the conduit 730. In this way, the conduit 730 is provided with a cylindrical cross-section.

The light source 750 is arranged to project light from the opening such that, in use, a beam of light is focussed onto the nipple of the user as the nipple is positioned into the opening. The light source 750 is provided proximal to a lens assembly 752 at the first end 724 of the conduit 730. That is, a lens assembly 752 is disposed between the light source 750 and the conduit 730.

The light source 750 is provided external to the conduit 730. That is, the light source is provided external to the wall 722 that encloses the conduit 730. In the example shown in FIG. 8, the lens assembly 752 is also arranged external to the conduit 730. The light source 750, lens assembly, 752, first end 724 and opening 732 are each arranged on the axis A.

In alternative examples, a lens assembly may integrally-formed with a portion of the wall. That is, the lens may be formed as a portion of the material of the conduit wall. Or a lens assembly may be provided within the conduit itself.

Referring additionally to FIGS. 9(a) and (b), the lens assembly 752 includes a first surface 754 and an opposing second surface 755. The first surface 754 is arranged to receive light projected from the light source 750. In the example show, the first surface 754 includes a recess to which the light source 750, provided as a light emitting diode (LED), is mounted.

The second surface 755 includes a lens arranged to focus light transmitted through the lens assembly from the first surface. In the example shown in FIG. 9, the lens is a Fresnel-type lens. The lens is a compact composite lens with a surface of a concentric series of simple lens sections.

The lens assembly 752 thereby focuses light received from the light source 750 prior to it entering the conduit 730. The light subsequently emitted from the lens is thereby focussed into a beam of light. Stated differently, with the light source 750 activated, light passing through the lens assembly 752 is focussed into a beam of light prior to entering the conduit 730.

The beam of light is projected from the opening 730 along axis A. The beam of light provides a first projected light region 791 having a cross-sectional area determined by the focussing of the lens assembly 752. The first projected light region 791 corresponds to light focussed by the lens assembly 752.

It should be noted that although the breast pump assembly 700 shown in FIGS. 8 and 9 is similar to the breast pump assembly of FIG. 2, it is not essential that a second portion of light from the light source 750 enters the wall 722 of the conduit 730. Optionally, with suitable configuration of the light source 750 and the lens assembly 752, a first portion of light L1, in the form of a beam of light, may be projected towards the opening 732 with negligible refraction of a second portion of light within the wall 722 itself.

Alternatively, the light from the light source 750 may be projected so as to form a first portion of light L1 in the form of a beam in addition to a second portion of light L2 refracted within the wall 722 itself. In this way, the second portion of light L2 is refracted within the breast receiving portion 740 towards its perimeter edge 742. The second portion of light L2 is then projected from the perimeter edge 742.

The breast pump assembly 700 further includes a port 760 provided proximal the first end 724. The port 760 is connectable to a negative pressure source for applying suction to a nipple received within the opening 732, as described with respect to examples herein.

A user desiring to use the breast pump assembly 700 to express milk positions the horn member 720 so as to align the nipple in substantially the same manner as when using the breast pump assembly 100 shown in FIG. 1. Thus, in use, the surface 799 illuminated by the beam of light projected from the opening 732 is a user's breast. The user aligns their nipple within the first projected light region 791. In particular, the user may align the centre of their nipple within the first projected light region 791

Maintaining the nipple within the first projected light region 791, the user moves the horn member 720 towards the breast until the nipple is within the opening 732. The user then continues moving the horn member 720, guiding the nipple into the opening 732, until the breast engages the breast receiving portion 740. The nipple and the breast are thus correctly engaged with the horn member 720 and ready to apply negative pressure to express milk.

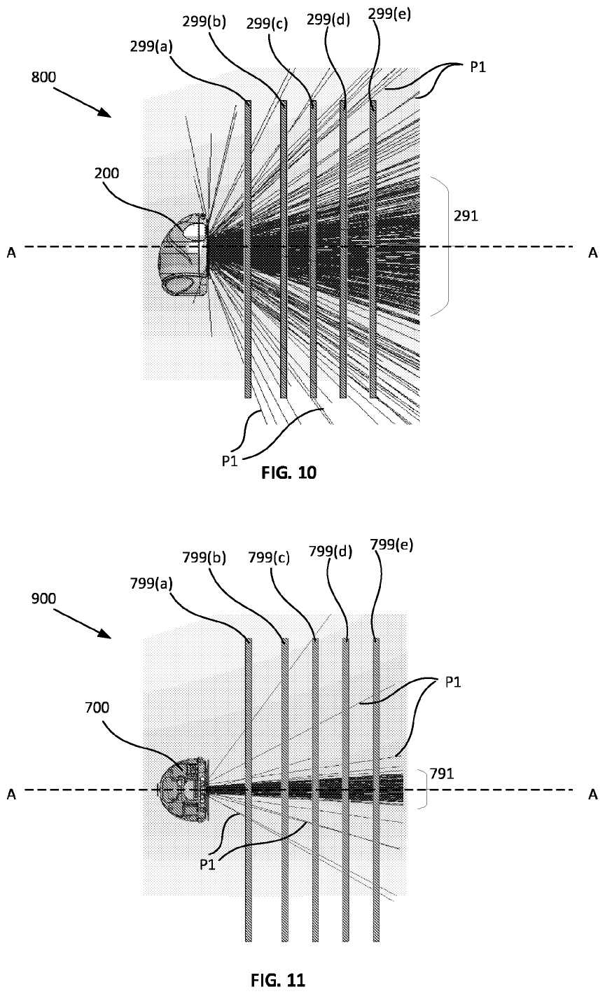

Referring now to FIG. 10, there is shown a computer-modelled ray tracing diagram 800 of a light source 250 within the example breast pump assembly 200 described with reference to FIG. 2. The modelling was undertaken using commercially-available Photopia® optical design software, in conjunction with a commercially-available Solidworks® Computer Aided Design (CAD) software package. The modelling estimates numerous paths P1 of individual light rays of the first portion of light L1 emitted by the light source 250. In this way, the modelling provides a guide to the expected focussing of light by the conduit 230 as it is projected from the opening 232 of the breast pump assembly 200.

In the example shown, the light source 250 is modelled as an RGB LED arranged to emit light along the longitudinal axis A of the conduit 230 within a 130-degree viewing angle. Accordingly, the first portion of light was projected from the opening 232 with a significant proportion of the individual paths P1 forming a first projected light region 291. The diameter of the first projected light region 291 was thereby measured at increasing distance from the opening 232 using a series of illuminance planes 299(a) to (e), arranged perpendicular to the axis A, as set out in Table 1.

TABLE 1

| | | Diameter of first projected |
|---|---|---|
| Plane | Distance from opening/mm | light region/mm |
| 299(a) | 50 | 80 |
| 299(b) | 100 | 115 |
| 299(c) | 150 | 150 |
| 299(d) | 200 | 185 |
| 299(e) | 250 | 220 |

Thus, the focussing of light by the conduit 230 of the breast pump assembly 200 effectively projects light within a predetermined region suitable to aid alignment of a nipple with the opening 232.

Referring now to FIG. 11, there is shown a computer-modelled ray tracing diagram 900 of a light source 750 within the example breast pump assembly 700 described with reference to FIGS. 8 and 9. The modelling was undertaken using the same optical design software and CAD software package as for the modelling described above.

The modelling estimates numerous paths P1 of individual light rays of the first portion of light L1 emitted by the light source 750. In this way, the modelling provides a guide to the expected focussing provided by the lens assembly 754 in projecting a beam of light from the opening 732 of the breast pump assembly 700.

In the example shown, the light source 750 is an RGB LED arranged to emit light along the longitudinal axis A of the conduit 730 within a 130-degree viewing angle. Light from the LED is modelled passing through a Fresnel-type lens assembly formed of a refractive acrylic polymer of 7 mm diameter. The second surface 754 is arranged 10 mm from the light source.

Accordingly, the first portion of light was projected from the opening 732 with a significant proportion of the individual paths P1 forming a first projected light region 791. The diameter of the first projected light region 791 was measured at increasing distance from the opening 732 using a series of illuminance planes 799(a) to (e), arranged perpendicular to the axis A, as set out in Table 2.

TABLE 2

| | | Diameter of first projected |
|---|---|---|
| Plane | Distance from opening/mm | light region/mm |
| 799(a) | 50 | 29 |
| 799(b) | 100 | 38 |
| 799(c) | 150 | 47 |
| 799(d) | 200 | 58 |
| 799(e) | 250 | 68 |

Thus, the focussing of light by the lens assembly 754 effectively provides a beam of light focussed along the longitudinal axis A of the conduit 730 suitable to aid alignment of the nipple with the opening 732. Furthermore, a very large proportion of paths P1 of light rays are focussed within the beam of light will, providing increased intensity which enables alignment even in bright lighting conditions.

It will be appreciated by persons skilled in the art that the above detailed examples have been described by way of example only and not in any limitative sense, and that various alterations and modifications are possible without departing from the scope of the invention as defined by the appended claims. Various modifications to the detailed examples described above are possible.

The opening of a horn member may be arranged with a breast receiving portion positioned in any suitable way such that the horn member receives the user's nipple in a way that provides comfort and effective expression. In this way, the opening may be provided centrally within, or off-centre to, the breast receiving surface. In certain examples, the opening may be arranged so that its longitudinal axis is perpendicular to the breast receiving surface at its location thereon. Alternatively, the opening may be arranged with its longitudinal axis at an angle away from a perpendicular to the breast receiving surface at its location thereon.

The light source in the examples shown above is positioned external to the conduit. That is, the light source is positioned external to the horn member and arranged so that emitted light is directed into the first end of the wall surrounding the conduit. In alternative arrangements, the light source may be positioned within the conduit, that is internal to the wall. In this way. the light source emits light directly into the conduit. Further alternatively, the light source may be integrated within the wall itself. Optionally, the integrated light source may be arranged within a recess of the wall which opens either to the conduit or to an external surface of the wall.

The light source may be a light emitting diode (LED), although any light source configurable to be arranged within a breast pump assembly may be used. If positioned internal to a wall of the horn member, that is within the conduit, then the light source should be unaffected by liquids, for example by using a water-resistant or water-proof light source.

A portion of the horn member, for example within the conduit or breast receiving portion, may be provided with different optical properties to the remainder of the horn member. Thus, a blocking portion may be provided by any portion of the horn member adapted to have different optical properties to enable it to cast a shadow within a projected light region. A perimeter edge, for example the perimeter edge 342 of the example shown in FIG. 4, may be considered a blocking region. Alternatively, any deformation such as a projecting edge or recessed feature may be provided to locally alter the refracting or reflecting properties of the horn member. A deformation may be provided at any suitable location on the horn member, including a wall of the conduit, an opening, a breast receiving portion or its perimeter edge so as to provide a shadow region or projected light region as is required.

It will be clear to a person skilled in the art that features described in relation to any of the examples described above can be applicable interchangeably between the different examples. The examples described above are to illustrate various features of the invention.

The following items are also herein disclosed:

1. A breast pump assembly, including:
a conduit including an opening adapted to receive a nipple of a user; and
a light source arranged to project light towards the opening, such that, in use, light is projected onto the nipple of the user as the nipple is positioned into the opening.

2. A breast pump assembly according to item 1, wherein the light source is arranged to project light from the opening.

3. A breast pump assembly according to item 2, wherein the conduit is configured to focus light projected from the opening within a predetermined region.

4. A breast pump assembly according to item 3, wherein the predetermined region has an area substantially equal to an area of the opening.

5. A breast pump assembly according to item 3 or item 4, wherein the conduit is configured to form a shadow region surrounding the predetermined region.

6. A breast pump assembly according to any preceding item, wherein the conduit is configured such that light from the light source is projected from the opening in an axial direction.

7. A breast pump assembly according to any preceding item, wherein the opening is arranged perpendicular to a central longitudinal axis of the conduit.

8. A breast pump assembly according to any preceding item, wherein the light source is provided external to the conduit.

9. A breast pump assembly according to any preceding item, further including a breast receiving portion extending from the opening of the conduit.

10. A breast pump assembly according to item 9, wherein the conduit and the breast receiving portion each include a wall having a refractive index such that a portion of light from the light source is refracted within the walls.

11. A breast pump assembly according to item 10, wherein the refractive index is within a range from 1.3 to 1.7.

12. A breast pump assembly according to item 10 or 11, wherein the wall of the conduit and the breast receiving portion is transparent or translucent.

13. A breast pump assembly according to any of items 10 to 12, wherein light refracted within the walls is projected from a perimeter edge of the breast receiving portion.

14. A breast pump assembly according to any of items 9 to 13, wherein the breast receiving portion flares outwardly from the opening of the conduit to a perimeter edge.

15. A breast pump assembly according to any of items 9 to 14, wherein the breast receiving portion is integrally formed with the conduit.

16. A breast pump assembly according to any preceding item, wherein the light source is adapted to selectively project light.

17. A breast pump assembly according to any preceding item, wherein the conduit includes a negative pressure supply port.

18. A breast pump assembly according to item 17, further including a negative pressure source fluidly coupled to the negative pressure supply port such that, when activated, the negative pressure source provides a negative pressure to the conduit.

19. A breast pump assembly according to item 19, wherein the negative pressure source is configured to be selectively activated, and wherein the light source is configured to deactivate when the negative pressure source is activated.

20. A breast pump assembly, including:
a breast receiving portion including an opening for receiving a breast of a user; and
a light source arranged to project light from the opening to form a projected light region having an area corresponding to an area of the opening.

21. A breast pump assembly according to item 20, wherein the breast receiving portion includes a perimeter edge around the opening, wherein the light source and the breast receiving portion are configured such that a portion of light from the light source is projected from the perimeter edge.

22. A breast pump assembly according to item 20 or item 21, wherein the breast receiving portion includes a wall having a refractive index such that a portion of light from the light source is refracted within the walls and projected from the perimeter edge.

23. A breast pump assembly according to any of items 20 to 22, wherein the perimeter edge is configured to form a ring shaped shadow around the projected light region.

24. A method of aligning a nipple within a breast pump assembly, the method including:
projecting light from an opening of a nipple receiving portion of a breast pump assembly to form a projected light region,
positioning the nipple of the user within the projected light region,
guiding the nipple into the opening of the nipple receiving portion whilst maintaining the nipple within the projected light region.

25. A method according to item 24, the method further including focussing the light projected from the opening within a predetermined region.

26. A method according to item 25, wherein the predetermined region has an area substantially equal to an area of the opening of the nipple receiving portion.

1. A breast pump assembly, comprising:
a conduit comprising an opening adapted to receive a nipple of a user; and
a light source arranged to project a beam of light from the opening such that, in use, the beam of light is focussed onto the nipple of the user as the nipple is positioned into the opening.

2. A breast pump assembly according to claim 1, wherein conduit comprises a central longitudinal axis and the beam of light is focussed along the central longitudinal axis.

3. A breast pump assembly according to claim 1, wherein the beam of light is focussed within a predetermined region.

4. A breast pump assembly according to claim 3, wherein the predetermined region has an area substantially equal to an area of the opening.

5. A breast pump assembly according to claim 1, wherein the conduit is configured such that the beam of light is projected from the opening in an axial direction.

6. A breast pump assembly according to claim 1, wherein the opening is arranged perpendicular to a central longitudinal axis of the conduit.

7. A breast pump assembly according to claim 1, wherein the light source is provided external to the conduit.

8. A breast pump assembly according to claim 1, wherein the beam of light is focused using a lens assembly.

9. A breast pump assembly according to claim 8, wherein the lens assembly comprises a Fresnel-type lens.

10. A breast pump assembly according to claim 8, wherein the conduit comprises a wall and the lens assembly is integrally formed with a portion of the wall.

11. A breast pump assembly according to claim 1, further comprising a breast receiving portion extending from the opening of the conduit.

12. A breast pump assembly according to claim 11, wherein the conduit and the breast receiving portion each comprise a wall having a refractive index such that a portion of light from the light source is refracted within the walls and, preferably, wherein the refractive index is within a range from 1.3 to 1.7.

13. A breast pump assembly according to claim 12, wherein light refracted within the walls is projected from a perimeter edge of the breast receiving portion.

14. A breast pump assembly according to claim 11, wherein the breast receiving portion flares outwardly from the opening of the conduit to a perimeter edge.

15. A breast pump assembly according to claim 11, wherein the breast receiving portion is integrally formed with the conduit.

16. A breast pump assembly according to claim 1, wherein the light source is adapted to selectively project light.

17. A breast pump assembly according to claim 1, wherein the conduit comprises a negative pressure supply port.

18. A breast pump assembly according to claim 17, further comprising a negative pressure source fluidly coupled to the negative pressure supply port such that, when activated, the negative pressure source provides a negative pressure to the conduit.

19. A breast pump assembly according to claim 18, wherein the negative pressure source is configured to be selectively activated, and wherein the light source is configured to deactivate when the negative pressure source is activated.

20. A breast pump assembly, comprising:
a breast receiving portion comprising an opening for receiving a breast of a user, wherein the opening comprises a longitudinal axis extending therethrough; and
a light source arranged to project light from the opening to focus a beam of light along the longitudinal axis.

21. A breast pump assembly according to claim 20, wherein the breast receiving portion comprises a perimeter edge around the opening, wherein the light source and the breast receiving portion are configured such that a portion of light from the light source is projected from the perimeter edge.

22. A breast pump assembly according to claim 20, wherein the breast receiving portion comprises a wall having a refractive index such that a portion of light from the light source is refracted within the walls and projected from the perimeter edge.

23. (canceled)

24. (canceled)

25. (canceled)
